(19) United States
(12) Patent Application Publication
(10) Pub. No.: US 2006/0161130 A1
(43) Pub. Date: Jul. 20, 2006

(54) Disposable absorbent article visually appearing similar to cloth underwear

(75) Inventors: Duane Kenneth Zacharias, Appleton, WI (US); Patsy Ann Krautkramer, Omro, WI (US); Gary Roland Woltman, Appleton, WI (US); David Glen Biggs, Neenah, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/036,403

(22) Filed: Jan. 14, 2005

(51) Int. Cl. A61F 13/15

(52) U.S. Cl. 604/385.26

(57) ABSTRACT

A disposable absorbent article is disclosed having a unitary structure with a waist opening and a pair of leg openings. The unitary structure includes a front region, a back region and a crotch region positioned between the front and back regions. The front and back regions have an interior surface and an exterior surface. A portion of at least one of the front and back regions has a visual appearance with a Shadow Index value of less than about 5 and/or a Gray-Level Percent Relative Standard Deviation value of less than about 70.

### BACKGROUND OF THE INVENTION

Disposable absorbent articles are available in many different sizes, styles and configurations and are designed for absorbing human exudate, such as urine and fecal matter. Most disposable absorbent articles are intended to be worn by infants, toddlers, or adults and are designed for single or temporary use. By “single or temporary use” it is meant that the disposable absorbent article will be disposed of after being used once instead of being laundered or cleaned for re-use, as is typical of regular cloth underwear. Examples of some disposable absorbent articles include an infant diaper, a child training pant, a feminine menstrual pant and an adult incontinent undergarment. Many of such disposable absorbent articles are similar in appearance, size and shape to regular cloth underwear except that they are formed from a variety of materials including absorbent and elastic materials. The absorbent materials allow the disposable absorbent article to absorb and retain body waste while the elastic material permits the disposable absorbent article to snugly conform to the anatomy of the user's torso.

Most manufacturers of such disposable absorbent articles are constantly looking to improve the appearance, aesthetics and normalcy of such articles to simulate cloth underwear. The reason for this is that the adult users of disposable absorbent articles have been preconditioned by years of wearing cloth underwear. “Cloth” underwear includes underwear formed from various natural and/or synthetic materials, including cotton, nylon, rayon, SPANDEX, as well as other materials known to those skilled in the art that are used to manufacture undergarments. Especially in adult incontinent undergarments, site and handling tests have indicated that women who use such products prefer that their disposable absorbent articles appear to be similar to cloth underwear. They are asking for a disposable absorbent article that both feels like and appears similar to cloth underwear.

Now a disposable absorbent article for absorbing human discharges has been invented that is constructed from a material that visually appears to be very similar to cloth underwear.

### SUMMARY OF THE INVENTION

Briefly, this invention relates to a disposable absorbent article having a unitary structure with a waist opening and a pair of leg openings. The unitary structure includes a front region, a back region and a crotch region positioned between the front and back regions. The front and back regions have an interior surface and an exterior surface. A portion of at least one of the front and back regions has a visual appearance with a Shadow Index of less than about 5 and/or a Gray-Level Percent Relative Standard Deviation of less than about 70.

### BRIEF DESCRIPTION OF THE DRAWINGS

FIG. 1 is a perspective view of a disposable absorbent article having a waist opening.

FIG. 2 is a plane view of the disposable absorbent article shown in FIG. 1. with an absorbent assembly secured to the inner surface of the front and back regions and showing a partial cut away view of the construction of the absorbent assembly.

FIG. 3 is a side view of the disposable absorbent article shown in FIG. 2 and taken along line 3-3.

FIG. 4 is a view of a person's buttock and rear torso when wearing the disposable absorbent article shown in FIG. 1 depicting a waist opening and a waist band secured to the exterior surface of the back region slightly below the waist opening.

FIG. 5 is an enlarged view of the encircled area shown on FIG. 4.

FIG. 6 is a schematic view of an apparatus and set-up for determining Shadow Index and Gray-Level Percent Relative Standard Deviation values of a material.

FIG. 7 is an image of a surface of a Hanes's Cotton Brief obtained using the apparatus and set-up shown in FIG. 6.

FIG. 8 is an image of a surface of a Kimberly-Clark Corporation's 2005 POISE panty obtained using the apparatus and set-up shown in FIG. 6.

FIG. 9 is an image of a surface of a SCA Hygiene Products Company's TENA DISCREET obtained using the apparatus and set-up shown in FIG. 6.

FIG. 10 is an image of a surface of a Kimberly-Clark Corporation's DEPEND Protective Underwear obtained using the apparatus and set-up shown in FIG. 6.

FIG. 11 is an image of a surface of a Tyco's ASSURANCE Underwear obtained using the apparatus and set-up shown in FIG. 6.

### DETAILED DESCRIPTION

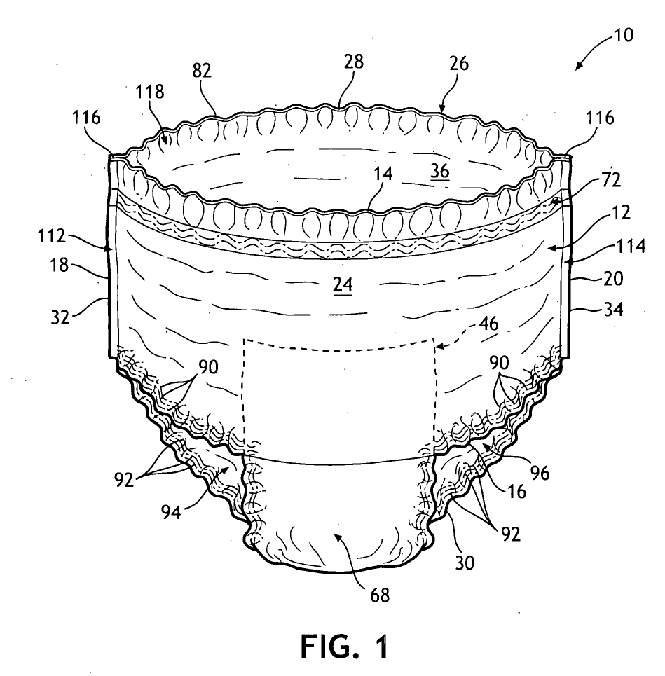

Referring to FIG. 1, a disposable absorbent article 10 is depicted that is designed for absorbing human exudate, such as urine and fecal matter. The disposable absorbent article 10 is intended to be worn by infants, toddlers, or adults, and is designed for a single or temporary use. The disposable absorbent article 10 is meant to be disposed of after being used once, instead of being laundered or cleaned for re-use. The disposable absorbent article 10 is designed to be pulled up around the user's torso without having to first open the absorbent article 10 in order to place it on a person's body. In FIG. 1, the disposable absorbent article 10 is shown as a Kimberly-Clark Corporation's 2005 POISE panty, which is an adult incontinent undergarment. The disposable absorbent article 10 is shown having its normal appearance just prior to being pulled up around a user's torso.

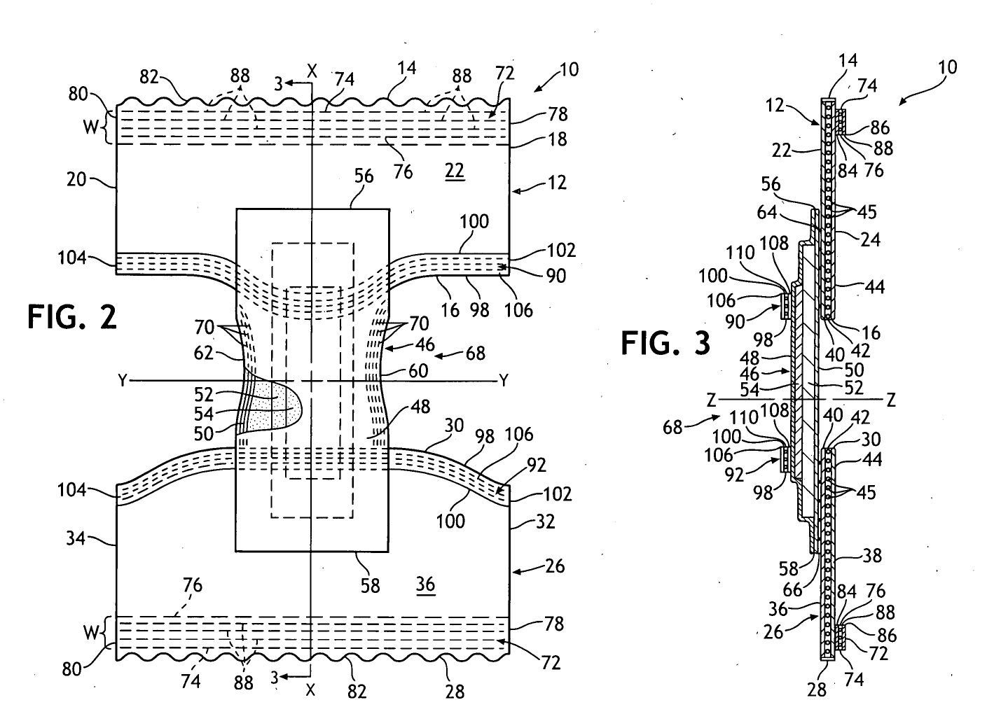

Referring to FIGS. 2 and 3, the disposable absorbent article 10 is depicted in an open configuration solely for the purpose of better showing the various components. The disposable absorbent article 10 in an open configuration has a longitudinal central axis X-X, a transverse central axis Y-Y and a vertical central axis Z-Z. The disposable absorbent article 10 includes a front region 12 having a first end 14 spaced apart from a second end 16 and a first side edge 18 spaced apart from a second side edge 20. Desirably, the first end 14 is scalloped. The front region 12 has an interior surface 22 and an exterior surface 24, see FIG. 3. The front region 12 can be constructed from a single layer or from two or more layers, such as a laminate. Desirably, the front region 12 is stretchable. By “stretchable” it is meant that the front region 12 can be lengthened, widened, or extended by applying a force, such as by pulling. Desirably, the front region 12 is also capable of retracting to or towards its original pre-stretched length. By “retraction” it is meant that the front region 12 can be shortened, or reduced in size or length. For example, a 1 inch (2.54 cm) strip of material can be stretched to about 2 inches (about 5 cm) and then when the stretching force is removed, the material will retract to or towards its original pre-stretched length of 1 inch (2.54 cm). The retracted length of the front region 12 is measured as the distance between the first and second side edges, 18 and 20 respectively. The retracted length is measured after the force required to stretch the material has been removed. A common means of making the front region 12 stretchable is to form it from an elastic component. By “elastic” is meant a material that is capable of quickly or immediately returning to or towards its initial form or state after deformation. Examples of stretchable materials include natural and synthetic rubbers, laminates containing at least one elastomeric layer, elastomeric films, spunbond, a spunbond laminate (SBL) or other material known to those skilled in the art. SBL is a material manufactured and commercially sold by Kimberly-Clark Corporation having an office at 401 North Lake Street, Neenah, Wis. 54956. Some stretchable materials and/or the process for making such stretchable materials are taught and described in U.S. Pat. Nos. 4,720,415; 5,336,545; 5,366,793; 5,385,775 and in Patent Publication 2002/0119722A1 dated Aug. 29, 2002, all of which are incorporated by reference and made a part hereof.

The disposable absorbent article 10 also includes a back region 26 having a first scalloped end 28 spaced apart from a second end 30 and a first side edge 32 spaced apart from a second side edge 34. Desirably, the first end 28 is scalloped. More desirably, the first scalloped end 14 of the front region 12 will be identical in size and configuration to the first scalloped end 28 of the back region 26. The back region 26 has an interior surface 36 and an exterior surface 38, see FIG. 3. The back region 26 can be constructed from a single layer or from two or more layers, such as a laminate. Desirably, the back region 26 is stretchable and also is capable of retraction. The same definitions recited above for the words “stretchable” and “retraction” apply to the back region 26. The retracted length of the back region 26 is measured as the distance between the first and second side edges, 32 and 34 respectively. The retracted length is measured after the force required to stretch the material has been removed.

The front and back regions, 12 and 26 respectively, are discontinuous from one another along the longitudinal axis X-X. The interior surfaces 22 and 36 of the front and back regions, 12 and 26 respectively, are in direct contact with the user's skin and are also sometimes referred to as the bodyside surfaces. The exterior surfaces 24 and 38 are situated opposite to the interior surfaces 22 and 36 and are spaced away from the user's skin. The exterior surfaces 24 and 38 are also sometimes referred to as the garment facing surfaces since they can be in direct contact with the inner surfaces of the user's outer clothing.

The front and back regions, 12 and 26 respectively, can be formed from a stretchable material. Desirably, the stretchable material is an elastic material. More desirably, the stretchable material is an elastic laminate that contains two or more layers wherein at least one of the layers is elastic. By “elastic layer” it is meant an elastic sheet, an elastic film, an elastic net-like material or a plurality of elastic strands aligned in a given plane. When a plurality of elastic strands is utilized, they can be positioned on a single carrier layer or be positioned between two or more outer layers. Each of the front and back regions, 12 and 26 respectively, can be formed such that essentially the entire region 12 and 26 is capable of being stretched and is also capable of being retracted. By “essentially the entire” it is meant that approximately about 90% to about 100% of the front region 12 is stretchable and retractable, and approximately about 90% to about 100% of the back region 26 is stretchable and retractable.

Referring now to FIG. 3, each of the front and back regions, 12 and 26 respectively, is shown being formed as a three-layer laminate. Each of the front and back regions, 12 and 26 respectively, includes a first layer 40, a second layer 42, and a third layer 44. The first and third layers, 40 and 44 respectively, are the outer layers and can be formed from the same material or from different materials. The first and third layers, 40 and 44 respectively, can be constructed from natural or synthetic fibers and can be a woven or non-woven material. The second or middle layer 42 has the elastic properties and is sandwiched between the first and third layers, 40 and 44 respectively. It should be noted that one or both of the first and third layers (outer layers) 40 or 44 can be made from an elastic material, if desired. The front and back regions, 12 and 26 respectively, can also be formed as a two-layer laminate with at least one of the layers being elastic, if desired.

The elastic laminate provides stretch and retraction in at least one direction. The stretch and retraction can be in one direction or in two or more directions. Desirably, the stretch and retraction of the front and back regions, 12 and 26 respectively, are in a direction approximately parallel to the transverse axis Y-Y. The transverse direction extends laterally across the torso from the left hip to the right hip. More desirably, the stretch and retraction of the front and back regions, 12 and 26 respectively, are in at least two directions, one direction being approximately parallel to the longitudinal axis X-X and the other direction being angled thereto. Most desirably, the stretch and retraction of the front and back regions, 12 and 26 respectively, are in multiple directions, or stated another way, in three or more directions extending over an arc of 360 degrees. The ability of the front and back regions, 12 and 26 respectively, to stretch and retract will provide a restrictive force during use of the disposable absorbent article 10 to ensure that it snugly conforms to the anatomy of the wearer's torso.

Desirably, the front and back regions, 12 and 26 respectively, are constructed from a stretch bonded laminate (SBL). Exemplary SBL materials are described in U.S. Pat. No. 4,720,415. In the stretch bonded laminate, the elastic core, or middle layer 42, is elongated before the two outer nonwoven layers 40 and 44 are attached. The attachment can be by an adhesive, by heat, by pressure, by a combination of heat and pressure, etc. Another material option for the front and back regions, 12 and 26 respectively, is a necked bonded laminate (NBL). The NBL material is also a three-layer laminate but the elastic core, or middle layer 42, is not pre-stretched prior to being attached to the two outer nonwoven layers 40 and 44. The outer layers 40 and 44 are necked stretched before the elastic core or middle layer 42 is attached to them. Exemplary NBL materials are described in U.S. Pat. No. 5,336,545. Other examples of elastomeric materials that can be used for the front and back regions, 12 and 26 respectively, include a continuous filament stretch bonded laminate (CFSBL) described in U.S. Pat. No. 5,385,775, a vertical filament laminate (VFL) described in Patent Publication 2002/0119722 A1 dated Aug. 29, 2002, a necked stretch bonded laminate (NSBL) and a necked thermal laminate (NTL). Combinations of the above materials can also be used.

It should also be noted that the front and back regions, 12 and 26 respectively, can be constructed from an elastic film that is capable of being stretched in at least one direction and desirably in both the machine direction and the cross-direction. Alternatively, the front and back regions, 12 and 26 respectively, can be formed from an elastic nonwoven that has a machine direction stretch and/or a cross-direction stretch. Extensible materials can also be used to form the front and back regions, 12 and 26 respectively, of the disposable absorbent article 10. Various other stretchable and elastic materials can be used which are known to those skilled in the art.

Still referring to FIG. 3, the front and back regions, 12 and 26 respectively, can alternatively be formed from two outer layers 40 and 44 with a plurality of elastic strands 45 sandwiched therebetween. The elastic strands 45 can be formed from LYCRA. The elastic strands 45 can be aligned approximately parallel to one another or be angled or skewed relative to one another. The elastic strands 45 can also be uniformly or randomly spaced apart from one another. The elastic strands 45 can vary in shape, size, configuration, and/or length. The diameter and/or cross-sectional configuration of the elastic strands 45, the decitex (weight in grams per 10,000 meters of a strand) of the elastic strands 45, and the tension imparted into the elastic strands 45 can all be varied to suit one's particular product needs. The elastic strands 45 can have a round, semi-circular, square, rectangular, oval or some other geometrical configuration. The elastic strands 45 can overlap, intersect or crisscross at least one other elastic strand 45. The various ways of positioning, orienting, and adhering the elastic strands 45 to the two outer layers 40 and 44 are well known to those skilled in the art.

Referring again to FIGS. 1-3, the disposable absorbent article 10 also includes an absorbent assembly 46 which is secured to the interior surfaces 22 and 36 of the front and back regions, 12 and 26 respectively. The absorbent assembly 46 includes a bodyside liner 48, an outer cover 50 and an absorbent 52 positioned therebetween. The absorbent 52 can include a superabsorbent material. Desirably, the bodyside liner 48 is liquid pervious and the outer cover 50 is liquid-impervious. A surge layer 54 can be optionally used, which is located between the bodyside liner 48 and the absorbent 52. The surge layer 54 can function to rapidly acquire and temporarily retain body fluid, such as urine, before it can be absorbed into the absorbent 52. Desirably, the surge layer 54 is also capable of wicking body fluid lengthwise and/or widthwise across its surface as well as directing the body fluid downward in a z-direction, toward the absorbent 52.

Referring to FIGS. 2 and 3, the absorbent assembly 46 has a first end 56, a second end 58, a first side edge 60 and a second side edge 62. The absorbent assembly 46 is shown secured to the interior surface 22 of the front region 12 approximate the first end 56 by an attachment 64. The absorbent assembly 46 is also secured to the interior surface 36 of the back region 26 approximate the second end 58 by an attachment 66. Alternatively, the absorbent assembly 46 can be secured to the exterior surfaces 24 and 38 of the front and back regions, 12 and 26 respectively, if desired. The absorbent assembly 46 can be secured to the front and back regions, 12 and 26 respectively, after each region has been stretched a predetermined amount. The amount that the front and back regions, 12 and 26 respectively, are stretched before the absorbent assembly 46 is attached can vary. By attaching the absorbent assembly 46 to the interior surfaces 22 and 36 of the front and back regions, 12 and 26 respectively, the absorbent assembly 46 is capable of being in direct contact with the wearer's body. This is beneficial in absorbing body fluids discharged by the wearer.

The attachments 64 and 66 can be a permanent type of attachment as well as being a removable or releasable attachment. Desirably, the attachments 64 and 66 are permanent attachments where they are not designed to be removed without destroying the bond. The attachments 64 and 66 can be formed by using glue, adhesive, ultrasonic bonds, thermal bonds, heat bonds, pressure bonds, heat and pressure bonds, or any other attachment mechanism known to those skilled in the art. The attachments 64 and 66 can also include a chemical bond or a mechanical fastener, such as by sewing with thread, using buttons and button holes, using snaps, by employing hook and loop fasteners, etc. A hook and loop fastener is generally considered a releasable attachment. One type of hook and loop fastener is VELCRO wherein a hook material is releasably engaged into a loop material.

The attachments 64 and 66 can be formed along a continuous line or over a surface area having a predetermined length and width. Alternatively, the attachments 64 and 66 can consist of intermittent point bonds that are spaced apart from one another. For example, the intermittent point bonds can be formed by using a hot or a cold melt adhesive or by forming ultrasonic bonds. Various bond formations can be used which are known to those skilled in the art. Desirably, the attachments 64 and 66 are formed using intermittent bonds because it allows the elastic material forming the front and back regions, 12 and 26 respectively, to gather the absorbent assembly 46 as the elastic contracts. This gathering feature causes the absorbent assembly 46 to remain in direct contact with the user's body prior to and during the time period that the absorbent assembly 46 is being insulted with body fluid.

The front and back regions, 12 and 26 respectively, can be stretched in a direction approximately parallel to the transverse axis Y-Y, or in any other direction or directions, before the absorbent assembly 46 is secured to it. The amount of stretch can vary. No stretch is required if one does not wish to do so. Desirably, the front and back regions, 12 and 26 respectively, are stretched at least about 5% from a relaxed state before the absorbent assembly 46 is secured thereto. Desirably, the front and back regions, 12 and 26 respectively, are stretched at least about 10% from a relaxed state before the absorbent assembly 46 is secured thereto. More desirably, the front and back regions, 12 and 26 respectively, are stretched at least about 25% from a relaxed state before the absorbent assembly 46 is secured thereto. Most desirably, the front and back regions, 12 and 26 respectively, are stretched from between about 25% to about 500% from a relaxed state before the absorbent assembly 46 is secured thereto. The front and back regions, 12 and 26 respectively, extend laterally beyond the first and second side edges, 60 and 62 respectively, of the absorbent assembly 46. The front region 12 also extends longitudinally beyond the first end 56 of the absorbent assembly 46 and the back region 26 extends longitudinally beyond the second end 58 of the absorbent assembly 46. This size configuration of the front and back regions, 12 and 26 respectively, allow them to elastically conform to the torso of the wearer.

Still referring to FIG. 2, when the absorbent assembly 46 is secured to the front and back regions, 12 and 26 respectively, a crotch region 68 is formed. The crotch region 68 separates the front region 12 from the back region 26 and is designed to cover the perineum area of the wearer. The crotch region 68 can cover a distance of a few inches in an infant diaper to several inches in an adult incontinence garment. For example, a crotch region 68 in an infant diaper may range from about 2 inches (about 5 centimeters (cm)) to about 10 inches (about 25 cm); while in an adult incontinence garment, the crotch region 68 may range from about 6 inches (about 15 cm) to about 20 inches (about 51 cm).

The absorbent assembly 46 can be stretchable or non-stretchable in relation to the front and back regions, 12 and 26 respectively. Desirably, the absorbent assembly 46 is non-stretchable in relation to the front and back regions, 12 and 26 respectively. By having the absorbent assembly 46 be non-stretchable in relation to the front and back regions, 12 and 26 respectively, it is meant that the absorbent assembly 46 will not appreciably stretch in the longitudinal or transverse directions. The reason for this is that the front and back regions, 12 and 26 respectively, are elastically stretchable and can extend and retract to snugly conform to the user's anatomy, especially to his or her torso. It is not necessary for the absorbent assembly 46 to stretch and/or retract to the same extent. The absorbent assembly 46 could alternatively be constructed with a pleated or folded construction, so as to be capable of being expanded in the longitudinal or transverse directions, if expansion of the absorbent assembly 46 is needed. The pleating or folding should take place before the absorbent assembly 46 is secured to the front and back regions, 12 and 26 respectively.

Normally, there is no need to have the absorbent assembly 46 gather as the front and back regions, 12 and 26 respectively, retract. However, the absorbent assembly 46 can be constructed and attached to the front and back regions, 12 and 26 respectively, in a way that will allow the absorbent assembly 46 to be gathered as the front and back regions, 12 and 26 respectively, elastically retract in a direction parallel to the transverse axis Y-Y. In either circumstance, the absorbent assembly 46 should remain over the perineum. As the absorbent assembly 46 receives body fluid and/or excrement discharged by the wearer, it will be displaced outward, away from the user's torso. The attachments 64 and 66 assure that the absorbent assembly 46 will be aligned over the perineum of the user while allowing the absorbent assembly 46 to move outward, in the z-direction, away from the torso as additional body fluid is received and retained.

Still referring to FIGS. 2 and 3, the absorbent assembly 46 also has at least one elastic member 70 positioned adjacent to and aligned approximately parallel to each of its first and second side edges 60 and 62. Each of the elastic members 70 is situated between the bodyside liner 48 and the outer cover 50. Each of the elastic members 70 provides a gasket to hold the side edges 60 and 62 of the absorbent assembly 46 against the user's body. Each of the elastic members 70 can be in the form of an elastic strand, ribbon or strip. Desirably, there are from about 2 to about 6 elastic members 70 positioned adjacent to each of the side edges 60 and 62. In FIGS. 2 and 3, three elastic members 70 are shown positioned adjacent to each of the side edges 60 and 62. The elastic members 70 can have a cross-sectional configuration that is round, square, rectangular or any other desired geometrical configuration. The elastic members 70 can be aligned parallel to the longitudinal axis X-X and should extend completely through the crotch region 68. The opposite ends of the elastic members 70 can terminate short of the front and back regions, 12 and 26 respectively, as shown in FIG. 2 or extend into the front and back regions, 12 and 26 respectively, if desired.

The disposable absorbent article 10 also includes a waist band 72 formed from an elastic material. In FIGS. 2 and 3, the waist band 72 is shown being secured to the exterior surfaces 24 and 38 of the front and back regions, 12 and 26 respectively. The waist band 72 can be attached in a semi-stretched condition. By “semi-stretched” is meant that the waist band 72 has not fully retracted to its original length. The waist band 72 can be secured to the front and back regions, 12 and 26 respectively, by using ultrasonics, by a thermal bond, by adhesive, by heat, by pressure, by a combination of heat and pressure, or a combination of any of the above. The waist band 72 can also be secured to the front and back regions, 12 and 26 respectively, by a chemical bond or by a mechanical attachment, such as by sewing with thread. Other means of securing the waist band 72 to the front and back regions, 12 and 26 respectively, are known to those skilled in the art.

The waist band 72 has a first edge 74, a second edge 76, a first side edge 78 and a second side edge 80. The first edge 74 is straight or linear in configuration so that it is visually distinguishable from the scalloped first ends 14 and 28. The first edge 74 is spaced apart from the scalloped first end 14 of the front region 12 and from the scalloped first end 28 of the back region 26. The first edge 74 can be spaced apart from the scalloped first ends 14 and 28 by at least about 2 millimeters (mm). Desirably, the first edge 74 will be spaced apart from the scalloped first ends 14 and 28, by at least about 3 mm. More desirably, the first edge 74 will be spaced apart from the scalloped first ends 14 and 28, by at least about 4 mm. Most desirably, the first edge 74 will be spaced apart from the scalloped first ends 14 and 28, by less than about 10 mm. The reason for this spacing is to provide an aesthetically pleasing and comfortable scalloped edge 82 on the disposable absorbent article 10. The scalloped edge 82 will provide the disposable absorbent article 10 with a similar appearance to that of cloth underwear.

Referring again to FIG. 2, the first and second side edges, 78 and 80 respectively, of the waist band 72 are aligned with the first and second side edges, 18 and 20 respectively, of the front region 12 and also with the first and second side edges, 32 and 34 respectively, of the back region 26. The waist band 72 has a width, denoted as (w), which can range from between about 3 millimeters (mm) to about 150 mm. Desirably, the width (w) of the waist band 72 ranges from between about 5 mm to about 100 mm. More desirably, the width (w) of the waist band 72 ranges from between about 10 mm to about 50 mm. Most desirably, the width (w) of the waist band 72 ranges from between about 12 mm to about 25 mm.

Referring again to FIGS. 2 and 3, the waist band 72 can be constructed from one or more layers. As depicted, the waist band 72 is constructed from two outer members 84 and 86 having an elastic material 88, such as one or more elastic strands, secured therebetween. Desirably, multiple elastic strands will be positioned between the two outer members 84 and 86. The outer members 84 and 86 can be formed from a non-elastic material. The non-elastic material can be various woven or non-woven materials. An example of a non-woven material that can be used to form one or both of the outer members 84 and 86 is polypropylene spunbond. Each outer member 84 and 86 can also be formed as a laminate, such as SBL, of two or more layers, if desired. The elastic strands can be formed from LYCRA. At least one of the elastic strands in the waist band 72 can extend from the first side edge 78 to the second side edge 80. Desirably, all of the elastic strands in the waist band 72 will extend from the first side edge 78 to the second side edge 80, as is depicted in FIG. 2. Alternatively, at least one of the elastic strands in the waist band 72 does not extend from the first side edge 78 to the second side edge 80. For example, one or more of the elastic strands in the waist band 72 can extend over only a portion of the distance located between the first and second side edges, 78 and 80 respectively.

The elastic strands can be aligned approximately parallel to one another or be angled or skewed relative to one another. The elastic strands can also be uniformly or randomly spaced apart from one another. The elastic strands can vary in shape, size, configuration, and/or length. The diameter and/or cross-sectional configuration of the elastic strands, the decitex (weight in grams per 10,000 meters of a strand) of the elastic strands, and the tension imparted into the elastic strands can all be varied to suit one's particular product needs. The elastic strands can have a round, semi-circular, square, rectangular, oval or some other geometrical configuration. The elastic strands can overlap, intersect or crisscross at least one other elastic strand. The various ways of positioning, orienting or securing the elastic strands to the outer members 84 and 86 are well known to those skilled in the art.

The number of elastic strands can range from 1 to about 50. The exact number of elastic strands that are present will depend on the width (w) of the waist band 72, the diameter of each elastic strand, the arrangement of the elastic strands on the outer members 84 and 86, etc. Desirably, from about 2 to about 25 elastic strands are present in the waist band 72. More desirably, from about 3 to about 20 elastic strands are present in the waist band 72. Most desirably, from about 3 to about 15 elastic strands are present in the waist band 72. In FIGS. 2 and 3, three elastic strands are depicted in the waist band 72.

Referring again to FIGS. 1-3, the disposable absorbent article 10 also includes a pair of leg bands 90 and 92. The leg band 90 is secured to the interior surface 22 of the front region 12 and the leg band 92 is secured to the interior surface 36 of the back regions 26. Each of the pair of leg bands 90 and 92 is positioned adjacent to one of the second ends 16 and 30 of the front and back regions, 12 and 26 respectively, and approximate leg openings 94 and 96, see FIG. 1. Each of the leg bands 90 and 92 has a first edge 98, a second edge 100, a first side edge 102 and a second side edge 104. The first edge 98 of the leg band 90 is a smooth outer edge that is aligned with the second end 16 of the front region 12. The first edge 98 of the leg band 92 is a smooth outer edge that is aligned with the second end 30 of the back region 26. Desirably, the first edge 98 of the leg band 90 is aligned flush with the second end 16 of the front region 12 while the first edge 98 of the leg band 92 is aligned flush with the second end 30 of the back region 26.

The first and second side edges, 102 and 104 respectively, of the leg band 90 are aligned with the first and second side edges, 18 and 20 respectively, of the front region 12. Likewise, the first and second side edges, 102 and 104 respectively, of the leg band 92 are aligned with the first and second side edges, 32 and 34 respectively, of the back region 26. Each of the pair of leg bands 90 and 92 can be constructed from two outer members 106 and 108 having an elastic material 110, such as one or more elastic strands, secured therebetween. Desirably, multiple elastic strands will be positioned between the two outer members 106 and 108. The outer members 106 and 108 can be formed from a non-elastic material as was explained above relative to the outer members 84 and 86 used to form the waist band 72. Likewise, the elastic material 110 can be the same as was explained above relative to the elastic material 88 used in the waist band 72. Normally, the leg bands 90 and 92 will have a narrower width than that of the waist band 72. Typically, from about 1 to about 6 elastic strands are present in each of the leg bands 90 and 92. Desirably, less than 4 elastic strands are present in each of the leg bands 90 and 92. In FIGS. 2 and 3, three elastic strands are present in each of the leg bands 90 and 92.

The elastic strands in the leg bands 90 and 92 are typically aligned approximately parallel to the second ends 16 and 30 of the front and back regions, 12 and 26 respectively. The second ends 16 and 30 are located adjacent to the crotch region 68 of the disposable absorbent article 10. The elastic strands in the leg bands 90 and 92 can be uniformly or randomly spaced apart from one another and are normally located within about 0.3 inches (about 0.76 cm) of the leg openings 94 and 96.

Referring now to FIGS. 1, 2 and 4, the absorbent assembly 46 of the disposable absorbent article 10 is capable of being folded transversely, approximate the transverse axis Y-Y. This folding enables the front region 12 to overlap the back region 26. When the absorbent assembly 46 is folded, the first and second side edges, 18 and 20 respectively, of the front region 12 will be aligned with the first and second side edges, 32 and 34 respectively, of the back region 26. In addition, the first and second side edges, 78 and 80 respectively, of the waist band 72 and the first and second side edges, 102 and 104 respectively, of the leg bands 90 and 92 will also be aligned with the first and second side edges 18 and 20 of the front region 12. After being folded, a pair of seams 112 and 114 are formed which join the front region 12 to the back region 26. The pair of seams 112 and 114 can form a permanent bond that is normally unbreakable during use or form a temporary bond that is designed to be easily broken when the disposable absorbent article 10 is to be removed from the user's torso and be discarded. Desirably, the pair of seams 112 and 114 will form a bond that is not easily broken or opened. The pair of seams 112 and 114 can be made using ultrasonics, heat, pressure, heat and pressure, adhesive, glue, or a combination of any of the aforementioned. In addition, the pair of seams 112 and 114 can be formed by a chemical bond or by a mechanical bond, such as by sewing with a thread. These and other types of bonds are well known to those skilled in the art. Desirably, the pair of seams 112 and 114 is formed using ultrasonic equipment.

Referring to FIG. 1, each of the pair of seams 112 and 114 has a scalloped end 116, which is located adjacent to the scalloped edge 82. Once the pair of seams 112 and 114 is formed, the disposable absorbent article 10 will become a unitary structure and acquire a pant-like appearance. The unitary structure will have a waist opening 118 adjacent to the scalloped edge 82. In addition, the pair of leg openings 94 and 96 will be spaced apart from the waist opening 118. Since the front and back regions, 12 and 24 respectively, are formed from a stretchable elastic material, the waist opening 118 and the pair of leg openings 94 and 96 can expand or retract in size to accommodate the anatomy of the user.

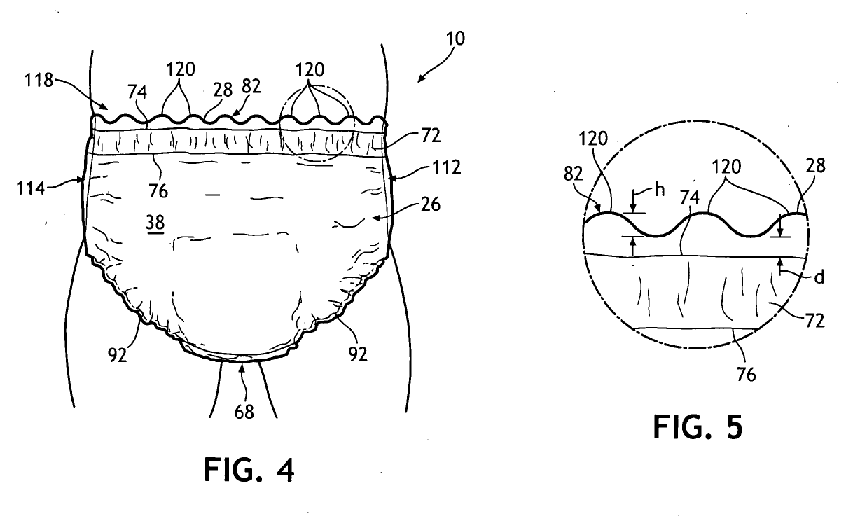

Referring now to FIGS. 4 and 5, the scalloped edge 82 contains of a series of semicircular curved projections 120 that form an ornamental border. The semicircular curved projections 120 have a height, denoted (h), which represents the distance between a peak and an adjacent trough. The height h should be less than about 5 millimeters (mm), desirably, less than about 4 mm, and more desirably, less than about 2 mm. The scalloped edge 82 acquires a very pleasing aesthetic appearance, which is similar to cloth underwear, when there are less than two semicircular curved projections 120 formed per inch when the front and/or back regions, 12 and/or 26 respectively, are in a relaxed condition. By “relaxed condition” it is meant that the front and/or back regions, 12 and/or 26, will not be subjected to a tension force. One curved projection 120 spans the distance from the bottom of one trough to the bottom of an adjacent trough. Desirably, there will be less than one and a half semicircular curved projections 120 formed per inch when the front and/or back regions, 12 and/or 26 respectively, are in a relaxed condition. More desirably, there will be one semicircular curved projection 120 formed per inch when the front and/or back regions, 12 and/or 26 respectively, are in a relaxed condition.

As noted above, the disposable absorbent article 10 is a unitary structure which includes the front region 12, the back region 26 and the crotch region 68. The front and back regions, 12 and 26 respectively, can be constructed from one or more materials, and desirably from a laminate, that visually appear to be very similar to cloth underwear. This visual appearance can be measured using the topography of the material itself to determine Shadow Index values and Gray-Level Percent Relative Standard Deviation values.

### Test Method

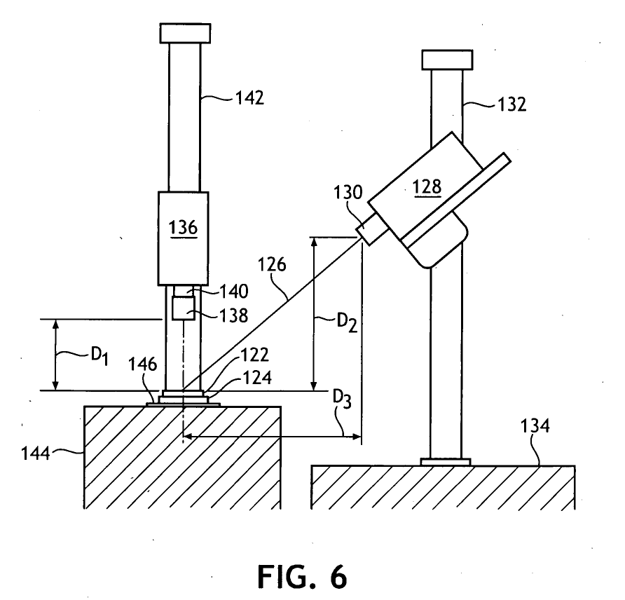

Referring to FIG. 6, the apparatus and set-up for determining the “Shadow Index” value and the Gray-Level Percent Relative Standard Deviation (GL % RSD) value of a sample material will be described in detail. The test method involves retaining the products, from which samples will be cut, at room temperature of between 68° F. to 72° F. for a time period of 24 hours. After the products have been acclimated, a sample 122 is cut out of either the front region or the back region of each product using scissors. The sample 122 is normally cut into rectangular shape and is done in such a way so that the longer dimension is parallel to the Y-Y direction, indicated in FIG. 2. The sample 122 is cut to approximately 8.5 inches (approximately 216 mm) by 5 inches (approximately 127 mm) in size. This size sample will allow three non-overlapping images to be acquired. If this size sample can not be cut from the product, then a smaller size sample can be cut. The minimum size sample 122 that can be cut out of the product to be tested will have a field of view size defined by the dimensions of approximately 68 mm by approximately 53 mm. For this smaller size sample, only one field of view can be obtained. For the smaller sample size used for obtaining a single image, as limited by the approximately 68 mm by approximately 53 mm field of view size, the longer dimension should be parallel to the X-X direction, as indicated in FIG. 2.

The sample 122 will have an interior surface and an exterior surface. The interior and exterior surfaces will correspond to the interior and exterior surfaces of the material as it is present in the unitary structure of the disposable absorbent article. Each sample 122 will be tested with the exterior surface facing up. Only one sample 122, dimensioned approximately 8.5 inches (approximately 216 mm) by 5 inches (approximately 127 mm), is needed to perform the test because three separate and non-overlapping images will be obtained from this size sample 122. However, when a smaller size sample is used, then more than one sample may be necessary in order to obtain three separate and non-overlapping images. For example, a smaller size sample having an optical field of view defined by the dimensions of approximately 68 mm by approximately 53 mm can yield one field of view image. Two additional samples, each having an optical field of view size defined by the dimensions of approximately 68 mm by approximately 53 mm, would be needed to obtain the remaining two images.

The sample 122 to be analyzed should exclude any portion of the waist band 72, the pair of leg bands 90 and 92, the absorbent assembly 46 or the pair of seams 112 and 114. After the sample 122 has been cut out of the front or back regions of the product, it is placed so that the Y-Y direction of the material, as indicated in FIG. 2, is aligned parallel to the light source. The sample 122 is then mounted onto an approximately 10 inch (approximately 254 mm) by 12 inch (approximately 305 mm) glass plate 124 with the interior surface of the sample 122 contacting the glass plate 124. The sample 122 is adhered to the glass plate 124 using pressure sensitive tape, such as transparent tape, to adhere each of the corners and sides of the sample 122. One method that works well is to first adhere two corners of one end of the sample 122 to the glass plate 124. If necessary, mild pressure can be applied to the sample 122 to remove any large wrinkles or bunching regions and the other two corners of the sample 122 are fastened onto the glass plate 124. Additional tape can be used along the sides of the sample 122 to ensure that the sample 122 remains in place. The sample 122 should lay flat with no macro-folds or wrinkles in the area of analysis because folds or wrinkles will produce an exaggerated shadow and generate erroneous data. The sample 122 should not be stretched any appreciable extent. After mounting the sample 122 onto the glass plate 124, the upper (exterior) surface of the sample 122 is prepared by painting the surface, via a top-quality camel's hair brush, with a 50:50 volume mixture of n-butyl alcohol and PENTEL Correction Pen liquid. This preparation will reduce light reflection and refraction as well as eliminate the affects that any graphics may present. If graphics show through after an initial coating, an additional coating should be applied to eliminate the visual appearance of the graphics.

Still referring to FIG. 6, the sample 122 is illuminated in a darkened room with a collimated light source 126 produced by a slide projector 128. The slide projector 128 used was a Kodak Ektgraphic slide projector (Model AF-3) having a lens 130. The slide projector 128, with its attached lens 130, was mounted on a support 132. In turn, the support 132 was attached to a base 134. The collimated light source 126 was adjusted to hit the top surface of the sample 122 at an angle of 30 degrees so that the shadow cast by the upper surface topography is generated from the X-X direction of the sample 122. The X-X direction of the sample 122 corresponds to the X-X direction of the disposable absorbent article 10, shown in FIG. 2. The resulting image of the sample 122, with shadows from the topography superimposed, is detected by a video camera 136 having a 35-millimeter adjustable lens 138. The adjustable 35-millimeter lens 138 was purchased from Nikon Instruments, Melville, N.Y. The detected image was then processed by an image analysis system to yield Shadow Index values and Gray-Level Percent Relative Standard Deviation (GL % RSD) values.

The video camera 136 used was a SONY® video camera (Model DXC-930P) with a synchronization and timing option (commonly called PAL format) and the red color channel was used. The adjustable 35-millimeter Nikon lens 138 was mounted on the video camera 136 via a 1:1 relay adaptor #C20047 (Century Optics, USA) 140. The 35-millimeter Nikon lens 138 had an f-stop setting of 4. The video camera 136 was mounted on a Polaroid MP-4 Land Camera (Polaroid Resource Center, Cambridge, Miss.) standard support 142. The support 142 was attached to a KREONITE macro-viewer 144 available from Kreonite, Inc., having an office in Wichita, Kans. An auto-stage 146, Model HM-1212, was placed on the upper surface of the KREONITE macro-viewer 144. The auto-stage 146 is a motorized apparatus known to those skilled in the analytical arts which was purchased from Design Components Incorporated, having an office in Franklin, Miss. The auto stage 146 was used to move the sample 122 in order to obtain three separate and distinct, non-overlapping images and associated measurements from the approximately 8.5 inch (approximately 216 mm) by 5 inch (approximately 127 mm) size sample 122.

The distance D, represents the distance between the upper surface of the sample 122 and the bottom of the lens 138. The distance D, was set to be approximately 33 centimeters (cm). The distance D₂ represents the vertical distance between the lens 130 attached to the slide projector 128 and the upper surface of the sample 122. The distance D₂ was set at 30 cm. The sample 122 was illuminated by the slide, projector 128. The distance D₃ represents the horizontal distance between a vertical line extending to the center of the video camera lens 138 and a vertical line extending to the center of the slide projector lens 130. The distance D₃ was set at 51 cm. These dimensions, combined with the video camera set-up, resulted in a field-of-view size of the sample 122 surface to be approximately 68 millimeters by approximately 53 millimeters. The slide projector 128 was connected to a POWERSTAT Variable Autotransformer, type 3PN117C, which was purchased from Superior Electric, Co. having an office in Bristol, Conn. The autotransformer is used to adjust the slide projector's 128 illumination level.

The image analysis system used to generate the data presented was a Quantimet 600 Image Analysis System available from Leica Microsystems, having an office in Wetzlar, Germany. The system was controlled and run by QWIN Version 1.06A software. The image analysis program ‘SHADIX3’ was used to acquire, process and measure images using Quantimet User Interactive Programming System (QUIPS) language. Alternatively, the SHADIX3 program could be used with a Quantimet 550 IW Image Analysis System which runs QWIN Version 2.4 software. The custom image analysis program is shown below.

NAME: SHADIDX3
PURPOSE: Measures the shadow index (i.e., area) and Gray-Level variation of a
textured material
AUTHOR: D. G. Biggs
DATE: Dec. 13, 2004
CONDITIONS: SONY 930-DXP w/ 35 mm adj. Nikon lens (f/4); Projected,
collimated light @ 30 deg. angle; PENTEL coating on samples (50:50 mix w/ n-
butanol; mounted on ¼″ glass plate; Front of fixture is 37 cm from front of
camera; fixture base is raised to 4th hole from bottom; macro-viewer pole position
= 80.0 cm
INITIALIZE VARIABLES
LFRAMECNT = 0
CALVALUE = 0.0929
FEATCOUNT = 0
TFEATCOUNT = 0
MFLDIMAGE = 3
SET-UP AND CALIBRATION
Open File (C:\EXCEL\DATA\ZACH1\SHADO1.XLS, channel #1)
Clear Accepts
Enter Results Header
File Results Header (channel #1)
File Line (channel #1)
Image Setup [PAUSE] (Camera 5, White 99.17, Black 88.23, Lamp 44.64)
Image frame (x 0, y 0, Width 736, Height 574)
Measure frame (x 52, y 62, Width 636, Height 511)
Calibrate (CALVALUE CALUNITS$ per pixel)
For (SAMPLE = 1 to 1, step 1)
PauseText (“Set up sample plate for analysis.”)
Image Setup [PAUSE] (Camera 5, White 99.17, Black 88.23, Lamp 44.64)
STAGE SCAN PARAMETERS
Stage (Define Origin)
Stage (Scan Pattern, 1 × 3 fields, size 87700.234375 × 72000.195313)
IMAGE ACQUISITION AND DETECTION
For (FIELD = 1 to 3, step 1)
ROUTINE TO STABILIZE LIGHT LEVEL
Y = 0
Z = 0
SP = 0
SIB = 0
P = 0
MGREYIMAGE = 0
FIELDS = 1000
TWICE = 0
Correlation GL Value for top 1% px Method, and SONY DXC930 = 187
For (LIGHT = 1 to 100, step 1)
Image Setup (Camera 5, White 99.17, Black 88.23, Lamp 44.64)
Live Image (into Image0)
Measure Grey (plane MGREYIMAGE,

histogram into GREYHIST(256), stats into GREYSTATS(3))
Selected parameters: Pixels, MeanGrey, Standard Deviation

A = GREYSTATS (2)
B = GREYSTATS (3)
D = A+B
For (X = 129 to 256, step 1)

Y = Y+(X*GREYHIST(X))
Z = Z+GREYHIST(X)

Next (X)
R = Y/Z
TP = GREYSTATS (1)
ONEPCTPX = .02 * TP
For (X = 256 to 1, step −1)

If (ONEPCTPX > SP)

P = GREYHIST(X)
SP = SP + P
SIB = SIB + (X * P)
If (ONEPCTPX < SP)

X =1

Endif

Endif

Next (X)
AVEGL = SIB / SP
E = AVEGL
Display (E, field width: 8, left justified, 1 digit after ‘.’, no tab follows)
If (E<200)

If (E>197)

TWICE = TWICE+1
If (TWICE=2)

Goto CONTINUE1

Endif

Endif

Endif
Y = 0
Z = 0
SP = 0
SIB = 0
Next (LIGHT)
END LIGHT STABILIZER ROUTINE
CONTINUE1:
IMAGE ACQUIRE AND DETECT
Image Setup (Camera 5, White 99.17, Black 88.23, Lamp 44.64)
Acquire (into Image 0)
ACQFILE$ = “C:\images\zach1\5i\”+STR$(FIELD)+“.TIF” (Note: This line indicates
the image file save location and will vary with application)
Write image (from ACQOUTPUT into file ACQFILE$, type TIF)
Detect (blacker than 31, from Image0 into Binary0 delineated)
IMAGE PROCESSING
Binary Amend (Open from Binary0 to Binary1, cycles 3, operator Disc, edge
erode on)
Binary Amend (Close from Binary1 to Binary2, cycles 2, operator Disc, edge
erode on)
Binary Amend (Open from Binary2 to Binary3, cycles 1, operator Disc, edge
erode on)
PERFORM FIRST MEASURMENTS (Shadow Index)
Measure field (plane MFLDIMAGE, into FLDRESULTS(3))

Selected parameters: Area, Perimeter, Area%

PERCAREA = FLDRESULTS (3)
AREA = FLDRESULTS (1)
PERIM = FLDRESULTS (2)
FACTOR1 = AREA/PERIM
File (“% Area = ”, channel #1)
File (PERCAREA, channel #1, 2 digits after ‘.’)
File Line (channel #1)
File (“Shado-index = ”, channel #1)
File (FACTOR1, channel #1, 3 digits after ‘.’)
File Line (channel #1)
Display Field Results (x −1, y 636, w 481, h 353)
Field Histogram #1 (Y Param Number, X Param Area%, from 0. to 50., linear, 25
bins)
Display Field Histogram Results (#1, horizontal, differential, bins + graph (Y axis
linear), statistics)

Data Window (735, 478, 540, 536)

SECOND MEASUREMENT (Grey-level variation)
Measure Grey (plane MGREYIMAGE,

Histogram into GREYHIST(256), stats into GREYSTATS(2))

Selected parameters: MeanGrey, Standard Deviation

MEANGREY = GREYSTATS (1)
SDGREY = GREYSTATS (2)
File (“Mean Gray (GL) = ”, channel #1)
File (MEANGREY, channel #1, 2 digits after ‘.’)
File Line (channel #1)
File (“GL Std. Dev. = ”, channel #1)
File (SDGREY, channel #1, 2 digits after ‘.’)
File Line (channel #1)
File Line (channel #1)
Binary Edit (Clear Binary3)
Stage (Step, Wait until stopped + 10 × 55 msecs)
Next (FIELD)
Next (SAMPLE)
Close File (channel #1)
End

Prior to testing the first sample 122, shading correction was performed using the QWIN software and a white, 803 Polaroid film positive (or equivalent white material) covered with an opaque, translucent film. Alternatively, other non-glossy white films or sheets could be used. The shading correction was performed using the ‘live’ mode. The system was also accurately calibrated using the QWIN software and a standard ruler with metric markings. The calibration was performed in the horizontal dimension of the video camera image.

After calibrating the system, the QUIPS routine SHADIX3 was executed via the QWIN software and this initially prompts the analyst to place the sample 122 within the field-of-view of the video camera 136. After positioning the sample 122 so the Y-Y direction is parallel to the light source and the sample 122 is properly aligned for auto-stage motion, the analyst will then be prompted to adjust the light level setting (via the POWERSTAT variable auto transformer) to register between Gray-Level readings of 197-200. During this process of light adjustment, the QUIPS routine SHADIX3 will automatically display the current Gray-Level value on the Quantimet 600 video screen.

After the light has been properly adjusted, the QUIPS routine SHADIX3 will then automatically acquire, detect, process and measure the image and the resulting shadows. The Gray-Level threshold value used in the routine to detect shadows is 31. The Gray-Level scale used on the Quantimet 600 system, or equivalent, is 8-bit and ranges from 0-255 (0 represents ‘black’ and 255 represents ‘white’). For shadow measurements, all regions in the image of the sample 122, that are at a Gray-Level of 31 or less, will be detected and measured by the SHADIX3 routine. For Gray-Level measurements, the entire Gray scale will be used.

The QUIPS routine SHADIX3 will then measure the Shadow Index value and the Gray-Level Percent Relative Standard Deviation values in the image. An explanation of each of the measurement parameters is described below:

Shadow Index—A derived parameter that combines shadow size and distribution across an entire image (Shadow Index=Shadow Area/Shadow Perimeter times percent area coverage). The “Shadow Area” is the area of an entire image that is covered by shadows at a predetermined detection threshold. The “Shadow Perimeter” is the distance around the outer boundary for the shadow area. For instance, if given two materials with equal percent area values, the material that has the larger sized individual shadows will also have a higher index value. This parameter gives an indication of both the distribution and level of shadow coverage.

Gray-Level Percent RSD (GL % RSD)—A normalized measure of a material's Gray-Level variation (GL % RSD=GL Std, deviation/Mean GL×100%). For example, a material with little or no noticeable topography (i.e., Hanes cotton briefs) will have a minimal GL % RSD value.

Data will be placed into corresponding histograms. The QUIPS routine SHADIX3 will then automatically move the auto-stage to position the sample 122 for next field-of-view to be measured. The analyst will again be prompted to re-adjust the light level, if needed, and the routine will again acquire, process, and measure the image. This process will be repeated until each of the three fields-of-view is measured. The data and basic statistics will then be available in an electronic format.

The whole image analysis and data collection process is repeated 3 times for each sample material (i.e., 3 replicate analyses). The mean data values are then determined from the 3 individual results obtained and these data can then be processed using Student's T analysis statistical methods to determine if significant differences exist between sample codes. Data are processed at the 90% confidence level.

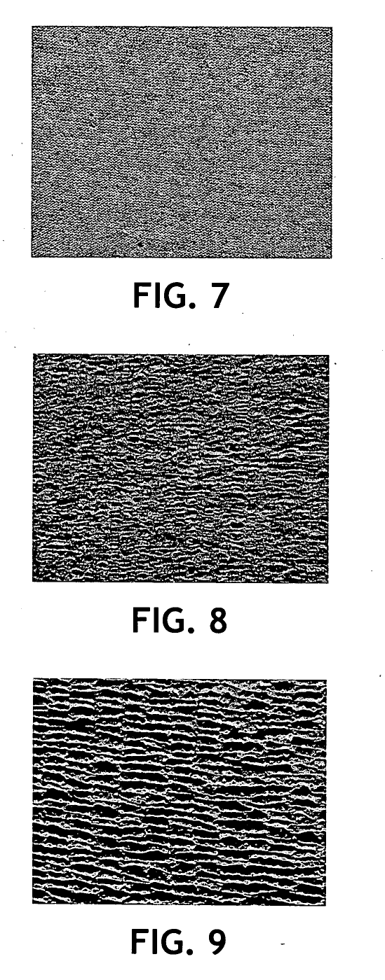

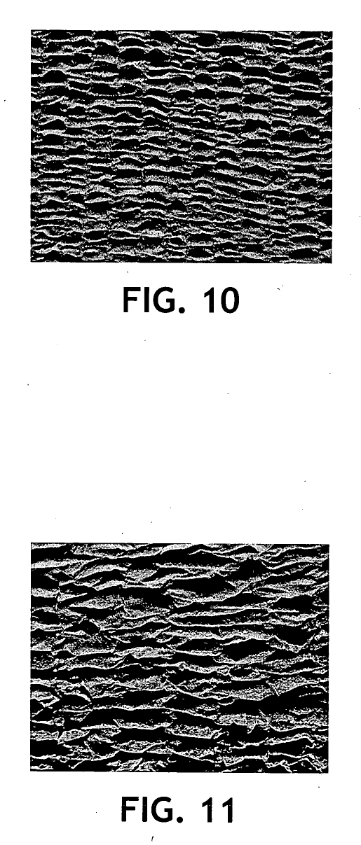

Referring now to FIGS. 7-11, images of the surface of a Hanes' Cotton Brief, a Kimberly-Clark Corporation's 2005 POISE panty, a SCA Hygiene Products Company's TENA DISCREET Underwear, a Kimberly-Clark Corporation's DEPEND Protective Underwear and a Tyco's ASSURANCE Underwear are shown. The Shadow Index and Gray-Level Percent Relative Standard Deviation values were calculated using the equipment shown in FIG. 6. The samples were all taken from the back region of each product. However, it should be noted that the samples could be taken from the front regions of each product if desired. Normally, the front and back regions of most products are constructed from the same material. In the Kimberly-Clark Corporation's 2005 POISE panty, the front and back regions are constructed of the same material. As can be seen from the images, the creation of shadows from the topographical differences lends itself nicely to image analysis measurements.

Table 1 below shows Mean Shadow Index and Gray-Level Percent Relative Standard Deviation values for these five products.

TABLE 1
Summary of mean values for the Shadow Index and
Gray-Level Percent Relative Standard Deviation Data

| | Mean | |
|---|---|---|
| | Shadow | Mean Gray-Level Percent |
| Sample ID | Index | Relative Standard Deviation |
| 1 - Hanes' Cotton Brief | 0.0 | 39.4 |
| 2 - K-C's 2005 POISE | 2.9 | 64.7 |
| panty | | |
| 3 - SCA's TENA DISCREET | 11.1 | 86.5 |
| 4 - K-C's DEPEND | 12.6 | 85.6 |
| Protective Underwear | | |
| 5 - Tyco's ASSURANCE | 15.1 | 85.6 |

Data were collected from three different fields-of-view (FOV) for each sample with the exception that for the Hanes' Cotton Brief wherein only two different fields-of-views were collected because the sample was of a smaller size. The size of the Hanes' Cotton Brief sample was approximately 6 inches (approximately 152.4 mm) by approximately 5 inches (approximately 127 mm). Student's T statistical comparisons of the confidences ranges at 90% is presented below. The separation between the ranges for the Kimberly-Clark Corporation's 2005 POISE panty (current invention) and the other materials is quite convincing.

The actual test values for all five samples are shown in Table 2 along with the calculated mean values.

TABLE 2

| Sample ID | Shadow Index | GL % RSD |
|---|---|---|
| Hanes' Cotton Brief | 0.00 | 40.33 |
| | 0.00 | 38.51 |
| Mean = | 0.00 | 39.42 |
| Std. Dev. = | 0.00 | 1.29 |
| K-C's 2005 POISE | 2.23 | 62.99 |
| panty | | |
| | 2.98 | 65.43 |
| | 3.34 | 65.64 |
| Mean = | 2.85 | 64.69 |
| Std. Dev. = | 0.56 | 1.47 |
| SCA's TENA | 12.30 | 87.66 |
| DISCREET | | |
| | 11.89 | 89.01 |
| | 9.04 | 82.90 |
| Mean = | 11.08 | 86.52 |
| Std. Dev. = | 1.77 | 3.21 |
| K-C's DEPEND | 14.36 | 87.38 |
| Protective | | |
| Underwear | | |
| | 11.19 | 84.40 |
| | 12.24 | 85.11 |
| Mean = | 12.60 | 85.63 |
| Std. Dev. = | 1.61 | 1.56 |
| Tyco's | 12.28 | 81.93 |
| ASSURANCE | | |
| | 17.60 | 89.01 |
| | 15.41 | 85.87 |
| Mean = | 15.10 | 85.60 |
| Std. Dev. = | 2.67 | 3.55 |

The data generated in Table 2 was subjected to a Student's T analysis at the 90% confidence level and yielded the ranges shown in the bar graphs depicted in Tables 3 and 4 below. Table 3 shows the ranges for the Shadow Index values and Table 4 shows the ranges for the Gray-Level Percent Relative Standard Deviation values.

TABLE 3
Student's T Confidence Limit
Ranges - Shadow Index
(90% Confidence)

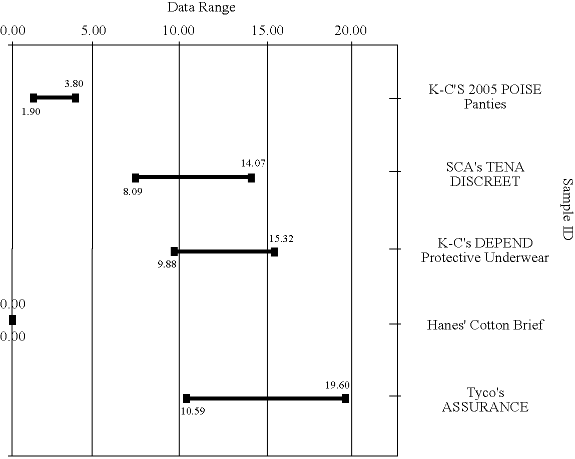

One can see from Table 3 that the Hanes' cotton brief registered at 0 for the Shadow Index. Kimberly-Clark Corporation's 2005 POISE panty had a visual appearance that was closest to the Hanes' cotton brief. The Kimberly-Clark Corporation's 2005 POISE panty had a Shadow Index range of from between about 1.9 to about 3.8. One can broadly interpret this Shadow Index range to be less than about 5. One can also interpret this Shadow Index range to extend from between about 1.0 to about 5.0. One can also interpret this Shadow Index range to extend from between about 1.5 to about 4.5. Another way of interpreting this range is to say that the Shadow Index of the Kimberly-Clark Corporation's 2005 POISE panty is less than about 4.

One can also see that the remaining three products all had a Shadow Index range of above 8. The higher the values, the more unlike the appearance of the Hanes' cotton brief. This data clearly indicates that the K-C's 2005 POISE panty, wherein the front and back regions were constructed from the same material, i.e. a laminate, had a Shadow Index value of less than about 5. This Shadow Index value indicates that the material used to form the front and back regions of the K-C's 2005 POISE panty has a visual appearance approaching the Hanes' cotton brief.

TABLE 4
Student's T Confidence Limit
Ranges - GL % RSD
(90% Confidence)

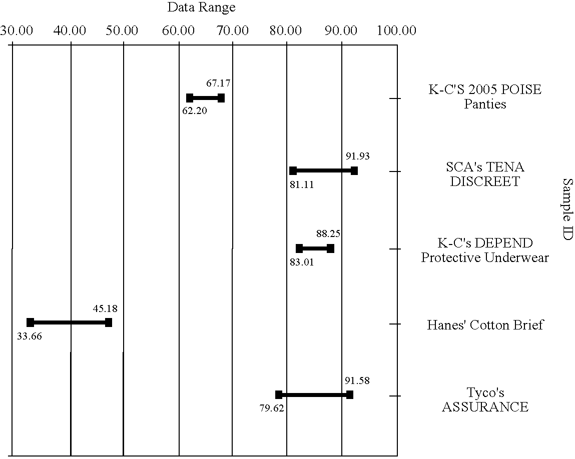

One can see from Table 4 that the Hanes' cotton brief had a Gray-Level Percent Relative Standard Deviation range of from between about 33.66 to about 45.18. Kimberly-Clark Corporation's 2005 POISE panty had a visual appearance that was closest to the Hanes' cotton brief. The Kimberly-Clark Corporation's 2005 POISE panty had a Gray-Level Percent Relative Standard Deviation range of from between about 62.2 to about 67.17. One can broadly interpret this Gray-Level Percent Relative Standard Deviation range to extend from between about 60 to about 70. One can also interpret this Gray-Level Percent Relative Standard Deviation range to extend from between about 61 to about 68. Another way of interpreting this range is to say that the Gray-Level Percent Relative Standard Deviation of the Kimberly-Clark Corporation's 2005 POISE panty is less than about 70.

One can also see that the remaining three products all had a Gray-Level Percent Relative Standard Deviation range of above 79.62. The higher the values, the more unlike the appearance of the Hanes' cotton brief. This data clearly indicates that The Kimberly-Clark Corporation's 2005 POISE panty, wherein the front and back regions were constructed from the same material, i.e. a laminate, had a Gray-Level Percent Relative Standard Deviation of less than about 70. This GL % RSD value indicates that the material used to form the front and back regions of the K-C's 2005 POISE panty has a visual appearance approaching the Hanes' cotton brief.

While the invention has been described in conjunction with a specific embodiment, it is to be understood that many alternatives, modifications and variations will be apparent to those skilled in the art in light of the aforegoing description. Accordingly, this invention is intended to embrace all such alternatives, modifications and variations that fall within the spirit and scope of the appended claims.

We claim:

1. A disposable absorbent article comprising a unitary structure having a waist opening and a pair of leg openings, said unitary structure including a front region, a back region and a crotch region positioned between said front and back regions, said front and back regions have an interior surface and an exterior surface, and a portion of at least one of said front and back regions having a visual appearance with a Shadow Index value of less than about 5.

2. The disposable absorbent article of claim 1 wherein said Shadow Index value is from between about 1.0 to about 5.0.

3. The disposable absorbent article of claim 2 wherein said Shadow Index value is from between about 1.5 to about 4.5.

4. The disposable absorbent article of claim 3 wherein said Shadow Index value is less than about 4.

5. The disposable absorbent article of claim 1 wherein said Shadow Index value is from between about 1.9 to about 3.8.

6. The disposable absorbent article of claim 1 wherein said exterior surface is liquid permeable.

7. The disposable absorbent article of claim 1 wherein said front and back regions are formed from a laminate including at least one elastic member.

8. The disposable absorbent article of claim 1 wherein said back region has a visual appearance with a Shadow Index value of less than about 4.

9. The disposable absorbent article of claim 1 wherein an absorbent assembly is secured to said interior surfaces of said front and back regions and said absorbent assembly includes a liquid pervious bodyside liner, a liquid-impervious outer cover, and an absorbent positioned therebetween.

10. A disposable absorbent article comprising a unitary structure having a waist opening and a pair of leg openings, said unitary structure including a front region, a back region and a crotch region positioned between said front and back regions, said front and back regions have an interior surface and an exterior surface, and a portion of at least one of said front and back regions having a visual appearance with a Gray-Level Percent Relative Standard Deviation value of less than about 70.

11. The disposable absorbent article of claim 10 wherein said Gray-Level Percent Relative Standard Deviation value is from between about 60 to less than about 70.

12. The disposable absorbent article of claim 11 wherein said Gray-Level Percent Relative Standard Deviation value is from between about 61 to about 68.

13. The disposable absorbent article of claim 12 wherein said Gray-Level Percent Relative Standard Deviation value is about 65.

14. The disposable absorbent article of claim 10 wherein said exterior surface is liquid permeable.

15. The disposable absorbent article of claim 10 wherein an absorbent assembly is secured to said interior surfaces of said front and back regions and said absorbent assembly includes a liquid pervious bodyside liner, a liquid-impervious outer cover, and an absorbent positioned therebetween.

16. A disposable absorbent article comprising a unitary structure having a waist opening and a pair of leg openings, said unitary structure including a front region, a back region and a crotch region positioned between said front and back regions, said front and back regions have an interior surface and an exterior surface, and a portion of at least one of said front and back regions having a Shadow Index value of less than about 5.0 and a Gray-Level Percent Relative Standard Deviation value of less than about 70.

17. The disposable absorbent article of claim 16 wherein said Gray-Level Percent Relative Standard Deviation value is from between about 60 to less than about 70.

18. The disposable absorbent article of claim 17 wherein said Shadow Index value is less than about 4.5.

19. The disposable absorbent article of claim 16 wherein said Shadow Index value is from between about 1.5 to about 4.5

20. A disposable absorbent article comprising a unitary structure having a waist opening and a pair of leg openings, said unitary structure including a front region, a back region and a crotch region positioned between said front and back regions, said front and back regions have an interior surface and an exterior surface, and a portion of at least one of said front and back regions having a Shadow Index value of less than about 5.0 or a Gray-Level Percent Relative Standard Deviation value of less than about 70.
